(19) United States
(12) Patent Application Publication
(10) Pub. No.: US 2013/0266992 A1
(43) Pub. Date: Oct. 10, 2013

(54) COMPOSITION FOR INCREASING SACCHARIFICATION EFFICIENCY OF BIOMASS, METHOD FOR INCREASING SACCHARIFICATION EFFICIENCY OF BIOMASS AND METHOD FOR PREPARING BIOETHANOL DERIVED FROM BIOMASS ON A LARGE SCALE USING CHAPERONE PROTEIN

(71) Applicant: KOREA ATOMIC ENERGY RESEARCH INSTITUTE, (US)

(72) Inventors: Byung Yeoup CHUNG, Jeollabuk-Do (KR); Seungsik LEE, Jeollabuk-Do (KR); Hyoungwoo BAI, Jeollabuk-Do (KR); Sung-Hyun HONG, Jeollabuk-Do (KR)

(73) Assignee: KOREA ATOMIC ENERGY RESEARCH INSTITUTE, Daejeon (KR)

(21) Appl. No.: 13/707,000

(22) Filed: Dec. 6, 2012

(30) Foreign Application Priority Data
Apr. 10, 2012 (KR) 10-2012-0037282

(51) Int. Cl. C12P 7/10

(52) U.S. Cl. CPC C12P 7/10
USPC 435/105; 435/165; 435/209

(57) ABSTRACT

The present invention relates to a novel method for increasing saccharification efficiency by adding chaperone protein during saccharification process, more precisely a composition for increasing saccharification efficiency of biomass containing cellulose using peroxiredoxin (Prx) protein and a method for increasing glucose yield by using the same. The conventional saccharification process has the disadvantage of decreasing cellulase activity, suggesting that the high priced saccharogenic enzyme has to be continuously supplied since the saccharogenic enzyme is losing its activity continuously by reducing sugar through the whole saccharification process. Therefore, to increase saccharification efficiency, the present invention provides a method characterized by increasing saccharification efficiency by preventing cellulase from losing its activity throughout the whole saccharification process by using chaperone protein, particularly Prx protein herein, together with cellulase. The method of the present invention, therefore, can overcome the disadvantage of the conventional saccharification process.

### CROSS REFERENCE TO RELATED APPLICATIONS

This application claims the benefit of Korean Patent Application No. KR 10˜2012˜0037282 filed on Apr. 10, 2012, the disclosure of which is incorporated herein by reference.

### REFERENCE TO SEQUENCE LISTING

This application contains a Sequence Listing submitted as an electronic text file named “12FPO_{—}10_{—}09_US_SequenceListing.txt”, having a size in bytes of 3 KB, and created on Apr. 10, 2012. The information contained in this electronic file is hereby incorporated by reference in its entirety pursuant to 37 CFR §1.52(e)(5).

### BACKGROUND OF THE INVENTION

1. Field of the Invention

The present invention relates to a novel method for increasing saccharification efficiency using chaperone protein, more precisely a method for increasing saccharification efficiency of cellulose by treating peroxiredoxin (Prx) having chaperone activity together with cellulase.

2. Description of the Related Art

The process for preparing ethanol energy from biomass is largely divided into two procedures; which are saccharification and fermentation. Hydrolysis indicates the conversion of cellulose molecular chain into sugar, which can be accomplished by using either enzyme or acid. The chemical method is to decompose cellulose by using acid, during which toxic hydrolysates are generated due to the harsh conditions of chemical hydrolysis, which makes the fermentation process difficult. Cellulose chain can also be decomposed into glucose by using the enzyme cellulase. This enzyme reaction is induced in the stomach of a ruminant such as cow or sheep at body temperature. At this time, diverse enzymes are working at each step. Major enzymes involved in this reaction are exemplified by endoglucanase, exoglucanase, and beta-glucosidase, etc. Saccharification is induced via hydrolysis through synergy effect produced by the above enzymes. The conventional method for saccharification has a problem of difficulty in pre-treatment for breaking lignin. Even more troublesome issue to be overcome is that cellulase has to be continuously supplied during the whole saccharification process because cellulase activity keeps getting down from the beginning of saccharification by reducing sugar generated during the process (the cellulase activity begins decrease 1 minute away from the process start and then only 2% of the initial activity remains 24 hours later). Considering high price of cellulase, the continuous supply of cellulase increases the cost of saccharification, requiring high financial support. Therefore, the conventional method has been limited in use industrially and economically owing to the said disadvantage.

Chaperone is the protein involved in protein folding (Korean Patent Publication No: 2003˜0085417). For example, once protein gets stress like heat shock, the original tertiary structure of the protein is denaturated, indicating the protein loses its function as a protein. Chaperone protein recognizes the denaturated protein and then helps it fold again (Korean Patent No: 10˜0476347). Molecular chaperone activity is largely divided into holdase activity and foldase activity. Holdase activity is working in the following processes: Once a protein is denaturated by the exposure on stress (oxidative stress or heat shock stress), some hydrophobic amino acid residues are exposed and denaturated protein fragments are aggregated irregularly to make aggregates. These aggregates are decomposed by protease and at this time chaperone protein (SHSPs, DnaJ) is conjugated to some of the denaturated hydrophobic amino acids to inhibit the aggregation and thus to make the protein come back to the original tertiary structure (Korean Patent Publication No: 10˜2011˜0014884, see FIG. 6).

In the meantime, foldase activity is working in the following processes; once a new protein is synthesized by ribosomes using mRNA as a template, protein folding is induced to make the protein has its original tertiary structure. At this time, chaperone protein (GroEL/ES, DnaK/J/E) is conjugated to the newly extended amino acid chain to form the authentic tertiary structure (see FIG. 7).

Peroxiredoxin (Prx) is one of the most representative chaperone proteins which is regulated by status of phosphorylation, oxidation-reduction, or oligomerization. There are three kinds of Prx proteins; which are classical 2-Cys Prx, atypical 2-Cys Prx, and 1-Cys Prx. These three kinds of Prx proteins share the equal basic catalytic mechanism, which is the oxidation of redox-active cystein in the active site into sulfenic acid by peroxide substrate. The difference between 2-Cys Prx and 1-Cys Prx lies in recycling of sulfenic acid by thiol. Particularly, 2-Cys Prx proteins are reduced by thiol such as glutathione, while 1-Cys Prx protein is reduced by ascorbic acid or glutathione in the presence of GST-.

It has been well-known that 2-Cys Prx protein has double enzyme activities of peroxidase and chaperone protein (Rhee S G et al. Free Radic Biol Med 38:1543-1552, 2005). It is also known fact that additional cystein, in addition to the above two cysteins, affects structural change of Prx protein. In particular, Prx PP1084 protein identified from Pseudomonas putida (KT2440) by the present inventors is a kind of 2-Cys Prx having two well-preserved active cysteins (Cys 51 and Cys 171). The said protein has additional cystein residue (Cys 112) in between the two active cysteins. The said Cys 112 plays an important role in regulating Prx protein activity. The activity of Prx PP1084 protein depends on its structure, that is, if it is a low-molecular polymer, peroxidase activity is strong, but if it is a high-molecular polymer, chaperone activity is strong. However, no reports have been made so far in relation to the use of the Prx protein having chaperone activity to increase saccharification efficiency.

Therefore, the present inventors studied to establish a novel method to increase saccharification efficiency. Particularly, the inventors measured saccharification efficiency when chaperone protein, particularly Prx protein, was used together with saccharogenic enzyme such as cellulase, a kind of hydrolase. As a result, saccharification efficiency was 24.9% in the absence of chaperone protein, while saccharification efficiency was significantly increased to 32.2% in the presence of chaperone protein. Such saccharification efficiency increasing effect of chaperone protein was observed when the ratio of saccharogenic enzyme such as cellulose showing strong chaperone activity to chaperone protein was at least 1:1, more preferably 1:1˜1:5. The present inventors completed this invention by confirming that chaperone protein can be effectively used for increasing saccharification efficiency by maintaining the activity of saccharogenic enzyme by using chaperone protein in the saccharification process.

### SUMMARY OF THE INVENTION

It is an object of the present invention to provide a method for increasing saccharification efficiency by maintaining the activity of the enzyme or enzyme composition by using chaperone protein, particularly peroxiredoxin (Prx) protein, to overcome the problem of the conventional method characterized by losing the activity of cellulase or saccharogenic enzyme composition including cellulase during the saccharification process.

To achieve the above object, the present invention provides a composition for increasing saccharification efficiency of biomass comprising chaperone protein and saccharogenic enzyme or saccharogenic enzyme composition as active ingredients.

The present invention also provides a composition for increasing saccharification efficiency of cellulose comprising chaperone protein and cellulase or saccharogenic enzyme composition containing cellulase as active ingredients.

The present invention further provides a method for increasing saccharification efficiency of biomass including the step of treating chaperone protein and saccharogenic enzyme or saccharogenic enzyme composition together to biomass.

The present invention also provides a method for increasing saccharification efficiency of cellulose including the step of treating cellulase and chaperone protein together to cellulose.

The present invention also provides a method for mass-production of bioethanol from biomass comprising the following steps:

1) inducing saccharification of biomass by treating chaperone protein and saccharogenic enzyme or saccharogenic enzyme composition together to biomass; and

2) producing ethanol by alcohol-fermenting monosaccharides generated by the saccharification of step 1).

In addition, the present invention provides a method for mass-production of bioethanol from cellulose comprising the following steps:

1) inducing saccharification of cellulose by treating chaperone protein and cellulase or saccharogenic enzyme composition containing cellulase together to cellulose; and

2) producing ethanol by alcohol-fermenting monosaccharides generated by the saccharification of step 1).

### ADVANTAGEOUS EFFECT

As explained hereinbefore, the present invention provides the effect of increasing saccharification efficiency by using chaperone protein in the process of saccharification of cellulose. More precisely, the conventional saccharification process using plant biomass has a disadvantage of losing the activity of saccharogenic enzyme, particularly cellulase, by reducing sugar produced during the saccharification process producing monosaccharides from polysaccharides including cellulose (the activity decreases from the beginning of the process and only 2% of the activity remains after 24 hour of saccharification). Therefore, the process has economical limitation since high priced saccharogenic enzyme has to be provided continuously during the process. The present inventors confirmed in this invention that when chaperone protein, particularly peroxiredoxin (Prx) was used together with saccharogenic enzyme during saccharification process, the chaperone protein could maintain the activity of saccharogenic enzyme throughout the whole process of saccharification, so that the saccharification efficiency was increased approximately 13%, compared with when chaperone protein was not used. Accordingly, the present inventors provided a novel use of chaperone protein for increasing saccharification efficiency. The present invention is also advantageous to increase glucose yield from plant biomass containing cellulose by using chaperone protein.

### BRIEF DESCRIPTION OF THE DRAWINGS

The application of the preferred embodiments of the present invention is best understood with reference to the accompanying drawings, wherein:

FIG. 1 is a graph illustrating the investigation of chaperone activity of peroxiredoxin (Prx) PP1084 protein obtained by the method described in Example 1, in which denaturation of malate dehydrogenase (MDH) by heat shock at 43° C. over Prx PP1084 concentration was illustrated with measuring OD₃₆₀.

FIG. 2 is a diagram illustrating the DNA sequence and amino acid sequence of Prx. PP1084 is typical 2-Cys Prx that contains well-preserved 2 active cysteins (red). There is another cystein (blue) in between these two active cysteins.

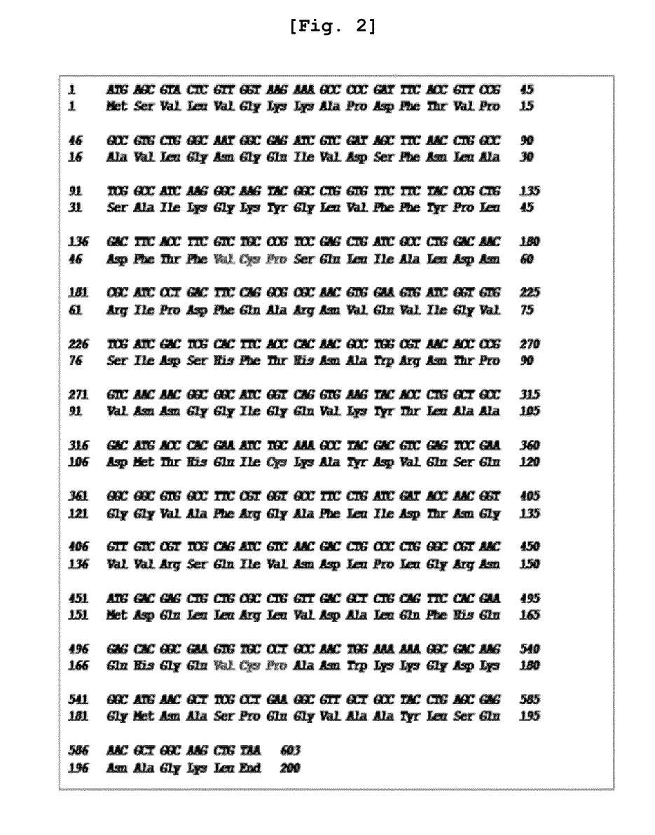

FIG. 3 is a graph illustrating the glucose yield from wheat stalk which was measured to compare saccharification efficiency between the negative control not treated with Prx protein and the experimental group treated with Prx PP1084.

FIG. 4 is a graph illustrating the glucose yield measured to compare saccharification efficiency among the groups treated with Prx protein and cellulase at different ratios.

FIG. 5 is a diagram illustrating the comparison of amino acid sequence of Prx PP1084 with those of 8 other organisms to investigate sequence homology.

FIG. 6 is a diagram illustrating holdase activity of Molecular chaperone activity.

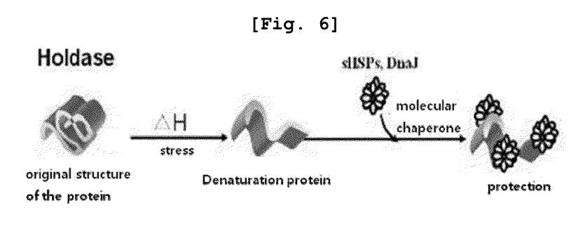

FIG. 7 is a diagram illustrating Foldase activity of Molecular chaperone activity.

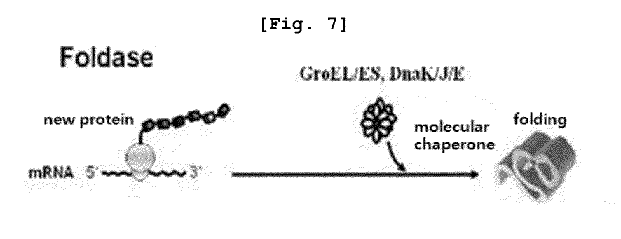

### DESCRIPTION OF THE PREFERRED EMBODIMENTS

Hereinafter, the present invention is described in detail.

The present invention provides a composition for increasing saccharification efficiency of biomass comprising chaperone protein and saccharogenic enzyme or saccharogenic enzyme composition as active ingredients.

The chaperone protein herein includes the protein having chaperone activity to prevent denaturation of tertiary structure by stresses including heat shock stress and oxidative stress, etc. The said protein is preferably peroxiredoxin (Prx) protein and more preferably Prx protein having the amino acid sequence represented by SEQ. ID. NO: 3, but not always limited thereto.

The said saccharogenic enzyme can be selected from the group consisting of cellulase, β-glucosidase, and xylanase, but not always limited thereto. When the saccharogenic enzyme is the enzyme mixture of three enzymes above, the ratio (cellulase:β-glucosidase:xylanase) is preferably 1˜10:1˜10:1˜10, more preferably 1˜10:1˜5:1˜5, and most preferably 6:1:1, but not always limited thereto.

The biomass herein includes cellulose, but not always limited thereto.

The saccharification process using the composition for increasing saccharification efficiency is characteristically performed not by acid saccharification but by enzymatic saccharification according to the conventional method known to those in the art. The ratio of the saccharogenic enzyme or the saccharogenic enzyme composition to chaperone protein is 1:1˜1:5, and more preferably 1:1˜1:2, but not always limited thereto.

In the process, saccharification is induced at 25˜45° C. with pH 4.5˜6.5, more preferably at 30˜40° C. with pH 4.5˜6.0, and most preferably at 37° C. with pH 5, but not always limited thereto. The saccharification time is preferably 12˜20 hours and more preferably 24˜96 hours, but not always limited thereto.

As explained in a preferred embodiment of the present invention, the present invention provides a composition for increasing saccharification efficiency of cellulose comprising chaperone protein and cellulase or saccharogenic enzyme composition containing cellulase as active ingredients.

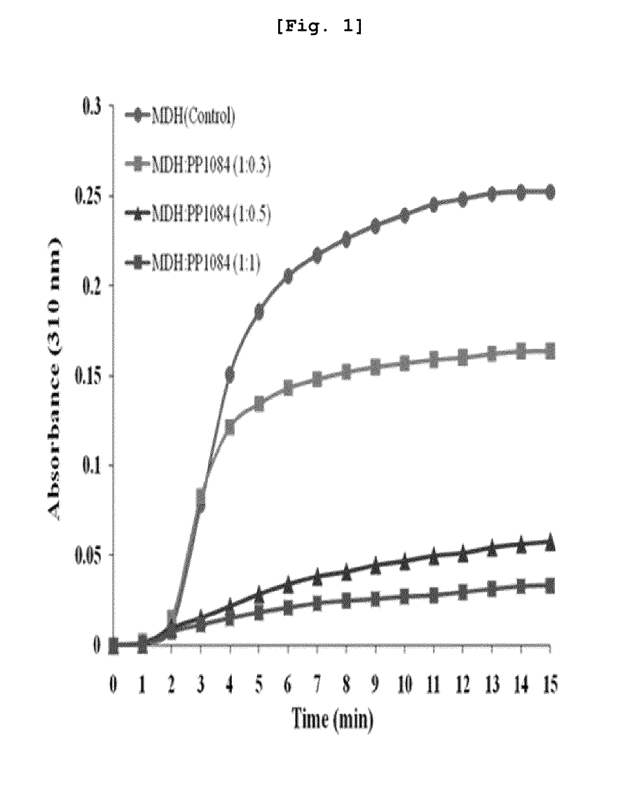

The present inventors obtained Prx protein gene fragment (PP1084) from Pseudomonas putida KT2440 genomic DNA via PCR using the forward primer represented by SEQ. ID. NO: 1 and the reverse primer represented by SEQ. ID. NO: 2, followed by cloning into pGEMT-easy vector. After cloning Prx PP1084 gene into the expression vector having T7 promoter system, pRETa, the transformant E. coli was constructed by using the said recombinant expression vector. The transformant E. coli was cultured and the culture solution was purified to obtain Prx PP1084 protein. To confirm chaperone activity of the obtained Prx PP1084 protein, the present inventors used heat-sensitive malate dehydrogenase (MDH). MDH starts to be denaturated when it is heated at 43° C., resulting in the aggregate. The level of denaturation can be measured by measuring OD₃₅₀. The denaturated protein having its tertiary structure broken demonstrates high OD₃₅₀, and OD₃₅₀ increases in proportion to MDH heating time. The present inventors added Prx PP1084 protein obtained in the above to MDH at different ratios, to which 43° C. heat shock was given continuously for 15 minutes, followed by measuring OD. As a result, in the presence of the Prx PP1084 protein having chaperone activity, the higher the concentration of the Prx PP1084 protein was, the lower the OD₃₅₀ went. This result suggested that heat-shock mediated MDH denaturation was inhibited by the Prx PP1084 protein. Such chaperone activity continued more than 15 minutes (see FIG. 1), indicating high chaperone activity of the Prx PP1084 protein.

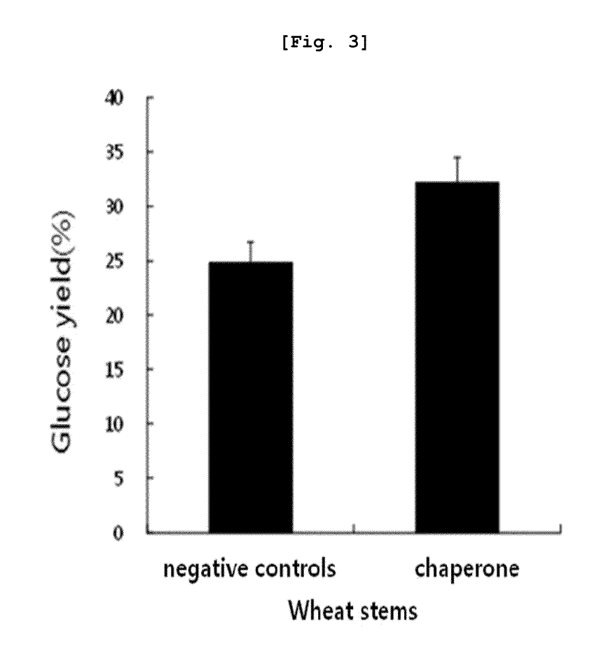

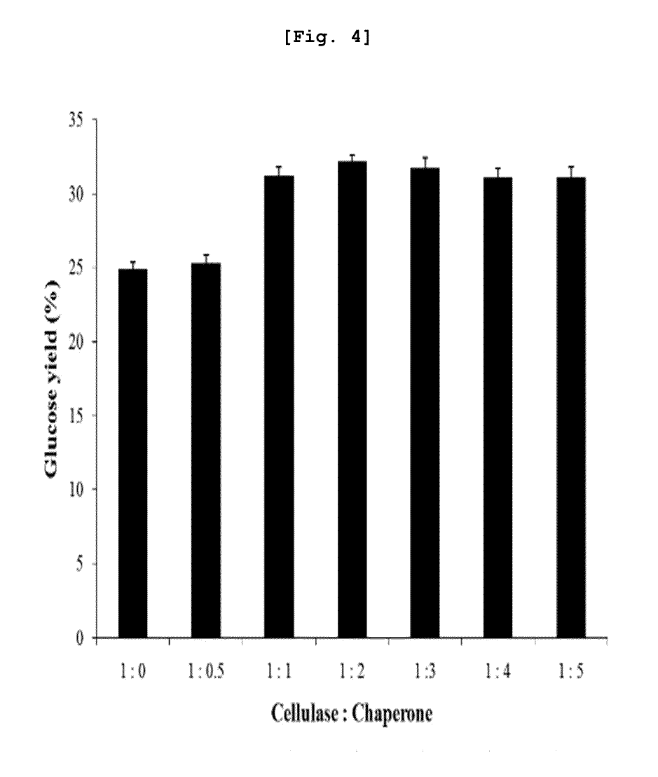

To investigate the effect of Prx protein on saccharification efficiency, the present inventors analyzed the content of ethanol and glucose yield in the presence of Prx protein. Particularly, wheat stalks were pulverized and treated with high temperature and high pressure to prepare biomass containing cellulose. As saccharogenic enzyme, the enzyme cocktail comprising cellulase, beta-glycosidase, and xylanase (6:1:1) was added to the prepared biomass along with Prx PP1084, followed by measuring glucose yield. As a result, glucose yield was approximately 13% higher in the experimental group treated with Prx PP1084 than in the negative control group not treated with Prx PP1084 (see FIG. 3). To determine the concentration of Prx PP1084 that could increase glucose yield significantly, the inventors tried many different ratios of enzyme cocktail to Prx PP1084, with which glucose yield was measured each time. As a result, the most preferable ratio of enzyme cocktail to Prx PP1084 to give most significant increase of glucose yield was determined as 1:1˜1:5 (see FIG. 4).

Therefore, the present inventors provided a novel use of Prx protein having chaperone activity for the effective increase of glucose yield, that is the increase of saccharification efficiency.

The present invention further provides a method for increasing saccharification efficiency of biomass including the step of treating chaperone protein and saccharogenic enzyme or saccharogenic enzyme composition together to biomass.

In this method, the chaperone protein can be any protein having chaperone activity that can maintain tertiary structure of the protein, which is preferably exemplified by peroxiredoxin (Prx) protein and more preferably Prx protein having the amino acid sequence represented by SEQ. ID. NO: 3, but not always limited thereto.

The saccharogenic enzyme in this method is selected from the group consisting of cellulase, beta-beta-glycosidase, and xylanase, but not always limited thereto. If the saccharogenic enzyme added herein is the enzyme cocktail composed of the said three enzymes, the ratio of them (cellulase:beta-beta-glycosidase:xylanase) is preferably 1˜10:1:1˜10, more preferably 1˜10:1˜5:1˜5, and most preferably 6:1:1, but not always limited thereto.

In this method, the biomass can contain cellulose, but not always limited thereto.

In this method, saccharification is characteristically performed not by acid saccharification but by enzyme saccharification according to the conventional method well known to those in the art. The ratio of cellulase to chaperone protein at this time is preferably 1:1˜1:5, and more preferably 1:1˜1:2, but not always limited thereto.

In this method, saccharification is induced at 25° C.˜45° C. with pH 4.5˜6.5, but not always limited thereto. Preferable time of saccharification is 12˜120 hours, but not always limited thereto.

The present invention also provides a method for increasing saccharification efficiency of cellulose including the step of treating cellulase and chaperone protein together to cellulose.

The present invention also provides a method for mass-production of bioethanol from biomass comprising the following steps:

1) inducing saccharification of biomass by treating chaperone protein and saccharogenic enzyme or saccharogenic enzyme composition together to biomass; and

2) producing ethanol by alcohol-fermenting monosaccharides generated by the saccharification of step 1).

In this method, the biomass of step 1) can include cellulose, but not always limited thereto.

In this method, the chaperone protein of step 1) can be any protein having chaperone activity, which is preferably exemplified by peroxiredoxin (Prx) protein and more preferably Prx protein having the amino acid sequence represented by SEQ. ID. NO: 3, but not always limited thereto.

In this method, the ratio of saccharogenic enzyme or saccharogenic enzyme composition of step 1) to chaperone protein is preferably 1:1˜1:5, but not always limited thereto.

In this method, the saccharogenic enzyme of step 1) is selected from the group consisting of cellulase, beta-glycosidase, and xylanase, but not always limited thereto.

In this method, the fermentation of step 2) is performed after treating yeast to monosaccharides in anaerobic condition at 25° C.˜45° C., more preferably at 30° C.˜35° C., but not always limited thereto.

As explained in a preferred embodiment of the present invention, the present invention provides a method for mass-production of bioethanol from cellulose comprising the following steps:

1) inducing saccharification of cellulose by treating chaperone protein and cellulase or saccharogenic enzyme composition containing cellulase together to cellulose; and

2) producing ethanol by alcohol-fermenting monosaccharides generated by the saccharification of step 1).

In this invention, the method of “Separate Hydrolysis and Fermentation (SHF)” wherein saccharification and fermentation are performed in different reactors separately, or the method of “Simultaneous Saccharification and Fermentation (SSF)” wherein saccharification and fermentation are performed in a same reactor can be used in order to produce bioethanol. Herein, the method of “Separate Hydrolysis and Fermentation” has the advantage of inducing reactions under the optimum condition for enzyme and yeast but has the disadvantage of accumulation of glucose over the reaction because the intermediate product and the final product, which are cellobiose and glucose, inhibit the enzyme reaction. To overcome the said disadvantage, a large amount of enzyme is required, which is not economic. In the meantime, in the method of “Simultaneous Saccharification and Fermentation”, as soon as glucose is produced by saccharification, yeast starts to act to eliminate the glucose via fermentation, resulting in the minimum accumulation of glucose in the reactor. That is, the inhibitory activity of the final product observed in the method of “Separate Hydrolysis and Fermentation (SHF)” can be prevented and at the same time hydrolytic activity of enzyme can be increased, which brings the effect of lowering costs of equipments and enzyme (comparatively low amount of enzyme is necessary in this method). In addition, contamination can be reduced because ethanol exists in the reactor. Therefore, it is preferred to use the method of “Simultaneous Saccharification and Fermentation” in this invention, but not always limited thereto.

The fermentation strain for the production of bioethanol herein can be yeast, a glucose-tolerant strain suitable for the fermentation at high glucose concentration, a thermo-tolerant strain suitable for the ethanol conversion at 40˜45° C., the optimum temperature for enzyme saccharification, a recombinant strain suitable for the simultaneous saccharification and fermentation to produce ethanol at high concentration with less amount of high-priced enzyme, for example Klebsiella oxytoca P2, Brettanomyces curstersii, Saccharomyces uvzrun, and Candida brassicae, which are generally known strains to those in the art, and more preferably can be Saccharomyces cerevisiae, but not always limited thereto.

As explained in a preferred embodiment of the present invention, the present invention provides, a method for increasing saccharification efficiency of cellulose including the step of treating peroxiredoxin (Prx) protein, and a mixture of cellulase, beta-glycosidase, and xylanase to cellulose.

In this method, the peroxiredoxin (Prx) protein was the amino acid sequence represented by SEQ. ID. NO: 3, but not always limited thereto.

In this method, the mixture comprises cellulase, beta-glycosidase, and xylanase mixed at the ratio of 6:1:1, but not always limited thereto.

In this method, the peroxiredoxin (Prx) protein and the mixture of cellulase, beta-glycosidase, and xylanase are treated at the ratio of one of 1:0, 1:0.5, 1:1, 1:2, 1:3, 1:4, and 1:5, but not always limited thereto.

In this invention, chaperone protein was used to increase saccharification efficiency of biomass. And it was confirmed that saccharification efficiency was increased by the method. Therefore, the method of the present invention having the effect of increasing saccharification efficiency can be used for the mass-production of bioethanol by alcohol-fermenting monosaccharides.

Practical and presently preferred embodiments of the present invention are illustrative as shown in the following Examples, Experimental Examples and Manufacturing Examples.

However, it will be appreciated that those skilled in the art, on consideration of this disclosure, may make modifications and improvements within the spirit and scope of the present invention.

### Example 1

### Peroxiredoxin (Prx) PP1084 Gene Cloning and Protein Purification

### <1-1> Peroxiredoxin (Prx) Gene Cloning

Prx protein gene was obtained from genomic DNA of Pseudomonas putida (KT2440) by polymerase chain reaction (PCR). The obtained gene was cloned into the cloning vector pEGMT-easy (Promega, Madison, Wis., USA), followed by sequencing analysis to confirm the gene. Then, the gene was sub-cloned in multi-cloning site of the expression vector pRSETa (Invitrogen, Carlsbad, Calif., USA).

First, PCR was performed for the cloning of PP1084 gene as follows. PCR reaction mixture was prepared with 10 ng of Pseudomonas putida KT2440 genomic DNA, 0.2 μM of dNTP, 20 μmol of the forward primer, 20 μmol of the reverse primer, and 1 unit of Taq polymerase, to which distilled water was added to make the total volume 20 μl. PCR was performed as follows; denaturation at 94° C. for 30 seconds, annealing at 50° C. for 45 seconds, extension at 72° C. for 45 seconds, 35 cycles from denaturation to extension, and final extension at 72° C. for 10 minutes. Primers used for PCR were as follows:

Pseudomonas putida KT2440 forward primer:
(SEQ. ID. NO: 1)
5′-ccgctcgagatgagcgtactc-3′;
and
Pseudomonas putida KT2440 reverse primer:
(SEQ. ID. NO: 2)
3′-cgagctcttacagcttgccagc-5′.

PCR product was obtained by PCR with the primers containing XhoI and ScaI sequences using genomic DNA of Pseudomonas putida KT2440 as a template. Prx gene was inserted in the cloning vector pGEMT-easy, followed by sequencing analysis to confirm the nucleotide sequence of the gene. The gene was sub-cloned in the multi-cloning site of the expression vector pRSETa.

For the expression and purification of Prx protein from the PP1084 gene inserted in the expression vector pRSETa, E. coli (KRX strain; Promega, USA) was transformed with the vector. Prx protein was over-expressed by using T7 promoter. Six histidines (His) were conjugated at N-terminal of Prx protein to purify Prx protein easily. To aid T7 promoter of pRESTa vector system working, T7 RNA polymerase was supplied to the host E. coli cells. KRX strain is the host E. coli in which T7 RNA polymerase supply is regulated by L-rhamnose. Prx PP1084 transformant clone was prepared to purify Prx protein.

### <1-2> Purification of Peroxiredoxin (Prx) Protein

In order to prepare Prx gene (PP1084) cloned in the expression vector pRSETa on a large scale as described hereinbefore, seed culture was inoculated [1/100 (v/v)] in 2 L Erlenmeyer flask containing 500 mL of LB (Luria-Bertani) medium, followed by shaking-culture at 37° C. at 150 rpm. Culture continued until OD₆₀₀ reached 0.4. Then, 20% L-rhamnose was added thereto (final conc.: 0.2%), followed by shaking-culture at 37° C. at 150 rpm. The cells induced by 1 mL of L-rhamnose were obtained by centrifugation at 4° C. at 6000 rpm for 10 minutes. The obtained cells were resuspended in binding buffer [20 mM Tris-HCl (pH 7.5), 0.5 M NaCl, 5 mM Imidazole] supplemented with 0.02% triton X-100, which were then stored at −20° C. 30 mL of the binding buffer was added to 400 mL of LB. The cells were lysed by using sonicator (VCX500; Sonics & Materials Inc., Newtown, Conn., USA). The lysate was centrifuged at 4° C. at 15000 rpm for 40 minutes to obtain supernatant. The supernatant was added to pre-equilibrated NTA-chelate resin (Peptron, Daejeon, Korea), followed by stirring in rotating wheel at 4° C. for at least 1 hour to induce binding. Upon completion of stirring, centrifugation was performed at 4° C. at 1000 rpm for 5 minutes to separate resin from supernatant. To the separated resin was added wash buffer [20 mM Tris-HCl (pH 7.5), 0.5M NaCl, 50 mM Imidazole] at the volume of approximately 5 times the resin, followed by stirring in rotating wheel at 4° C. for at least 1 hour to eliminate the supernatant. This process was repeated 5 times. To the resin was added elution buffer [20 mM Tris-HCl (pH 7.5), 0.5M NaCl, 200˜400 mM Imidazole] at the volume of 0.5˜1 times the resin, followed by stirring in rotating wheel at 4° C. for at least 1 hour. Then, the supernatant was obtained. Collecting the supernatant by adding elution buffer was repeated three times. The obtained eluted fractions were transferred into membrane tube, followed by dialysis by using 1 L of 50 mM Hepes (pH 8.0) three times. Concentration was performed by using centricon (Millipore, Carrigtwohill, Co. Cork. Ireland), which was then stored at −80° C. until use.

### Example 2

### Comparison of Homology Between the Amino Acid Sequence of Peroxiredoxin (Prx) PP1084 Protein and Those of Prx-Like Proteins of Other Organisms

The following experiment was performed to compare homology between the peroxiredoxin (Prx) PP1084 purified above and other peroxiredoxin proteins found in other species.

The amino acid sequence of PP1084 identified by using mass spectrometer was blasted with amino acid sequences of other organisms registered in NCBI.

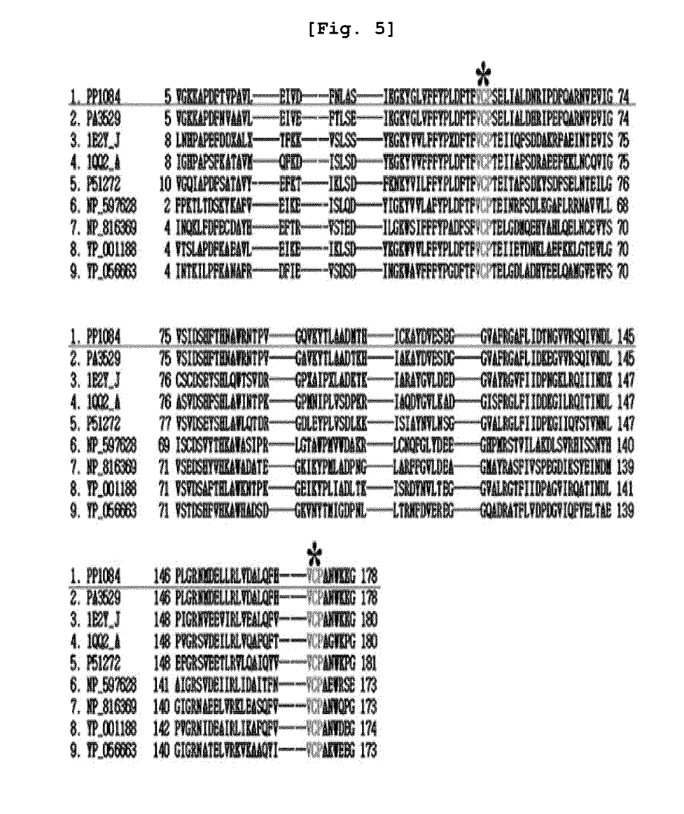

As a result, the amino acid sequence of PP1084 demonstrated high homology with amino acid sequences of 8 other organisms. Particularly, it was confirmed that they shared similarity in active cysteine motif [VCP motif (Val-Cys-Pro motif)] containing active cysteine (FIG. 5). The above 8 other proteins were confirmed to be the proteins known as the antioxidant proteins alkyl hydroperoxide reductase C (AhpC), thioredoxin-dependent peroxiredoxin (TPx), and peroxiredoxin (Prx).

### Example 3

### Measurement of Chaperone Activity of Peroxiredoxin (Prx) PP1084 Protein

The following experiment was performed to measure chaperone activity of peroxiredoxin (Prx) PP1084 protein. In general, the activity of molecule chaperone is divided into holdase activity and foldase activity, as described hereinbefore. In this experiment, specifically holdase activity of chaperone was analyzed.

To analyze holdase activity, 43° C. heat stress was used as a stress. And heat-sensitive malate dehydrogenase (MDH) was used as an enzyme. Once heated at 43° C., MDH was denaturated and turned into aggregates. The denaturation level could be measured by measuring OD₃₄₀. Thus, OD₃₄₀ was increased in proportion to heating hours of MDH. Total volume of reaction mixture was 300 μl. MDH in 50 μM of HEPES (pH8.0) or MDH added with Prx PP1084 protein having chaperone activity was heated at 43° C. for 15 minutes. During which, OD₃₄₀ was measured by using spectrophotometer (EVOLUTION 300 UV-VIS spectrophotometer, Thermoscientific, Worcester, Mass., USA). Prx PP1084 free MDH was used for the negative control. In the meantime, MDH added with Prx PP1084 at different ratios, as shown in Table 1, was used for the experimental group.

TABLE 1

| | Negative | | Experimental | | Experimental | | Experimental | |
|---|---|---|---|---|---|---|---|---|
| | Control | | Group 1 | | Group 2 | | Group 3 | |
| Ratio of MDH | 1:0 | | 1:0.1 | | 1:0.5 | | 1:1 | |
| to Prx PP1084 | | | | | | | | |
| (molar ratio) | | | | | | | | |
| Prx PP1084 | 0 | | 1.33 | μl | 6.6 | μl | 13.3 | μl |
| (1 μg/uL) | | | (1.33 | μg) | (6.6 | μg) | (1.33 | μg) |
| MDH | 6.5 | μL | 6.5 | μL | 6.5 | μL | 6.5 | μL |
| (total 20 μg) | | | | | | | | |
| 1M | 15 | μL | 15 | μL | 15 | μL | 15 | μL |
| HEPES(pH8.0) | | | | | | | | |
| (final conc.: | | | | | | | | |
| 50 mM) | | | | | | | | |
| DW | 278.5 | μL | 277.17 | μL | 271.9 | μL | 265.2 | μL |
| Total | 300 | μL | 300 | μL | 300 | μL | 300 | μL |

As a result, when 43° C. heat stress was given, OD₃₄₀ was decreased Prx PP1084 dose-dependently. The chaperone activity was maintained even against 42° C. heat stress at least for 15 minutes (FIG. 1).

### Example 4

### Saccharification Efficiency of Biomass Increased by Peroxiredoxin (Prx) Protein

In order to investigate the effect of peroxiredoxin (Prx) protein obtained in Example 1 on saccharification efficiency, the following experiment was performed.

### <4-1> Preparation of Biomass Sample

To investigate the correlation of saccharification efficiency and Prx protein, wheat stalks were pulverized and separated by using 5 different sized sieves (less than 200 μm, 200˜510 μm, 510˜710 μm, 710˜1000 μm, and more than 1000 μm). Dilute sulfuric acid (3%) was added to the separated samples filtered by each sieve, followed by high temperature/high pressure treatment at 121° C. for 30 minutes.

### <4-2> Increase of Saccharification Efficiency of Biomass by the Addition of Irradiated Prx

To measure saccharification efficiency of biomass over the addition of Prx PP1084 protein, enzyme cocktail was prepared by mixing R-10 cellulase (Onozuka, Japan), beta-glucosidase (Sigma, USA), and xylanase (Sigma, USA) at the ratio of 6:1:1 (160 μg/mL:27 μg/mL:27 μg/mL), to which Prx PP1084 protein prepared in Example 1 was added at the ratios of 1:0, 1:0.5, 1:1, 1:2, 1:3, 1:4, and 1:5 (molar ratio, when the ratio of cellulase to PP1084 was 1:1, 800 μg of cellulase and 200 μg of PP1084 protein were used), followed by comparing each saccharification efficiency. Negative control group was treated equally but without Prx PP1084 protein. Saccharification efficiency was also measured under the same condition as described above.

Saccharification efficiency of each sample was measured as follows. First, 5 g of wheat stalk powder and 487 mL of citrate buffer [citric acid 1-hydrate 10.57 g/L (pH 5.0)] were loaded in 1 L Erlenmeyer flask, which was stirred at 37° C. with 140 rpm for 1 hour to make the optimum condition for cellulase cocktail. 13 mL of cellulase cocktail was added to 487 mL of the reaction mixture to prepare 500 mL of saccharification solution, which was stirred at 37° C. with 140 rpm for 96 hours to induce saccharification of the wheat stalk powder. Upon completion of saccharification, the reaction mixture was centrifuged (6000 rpm, 10 min., 4° C.) to precipitate the remaining powder, which was then eliminated. Supernatant was obtained. The concentrations of glucose and xylose (mg/mL) in the reaction mixture were measured by using standard curve. The reaction mixture having low concentration of glucose was heated at 40° C. for evaporation and the remaining moisture was freeze-dried, leading to the complete powderization. To add saccharified powder to ethanol fermentation process by absolute glucose concentration, absolute glucose concentration (g/g) of the pulverized glucose mixture was first calculated.

### <4-3> Increase of Ethanol Content of Biomass by the Addition of Irradiated Prx

For alcohol-fermentation, Saccharomyces cerevisiae KCCM11626 strain was used in this invention. S. cerevisiae cells were inoculated in the test tube containing 10 mL of YPG complete medium [(glucose: 5 g, yeast extract: 0.5 g, peptone: 0.5 g, MgSO₄: 0.1 g, K₂HPO₄: 0.1 g/100 mL (pH 5)], followed by pre-culture at 30° C. with 180 rpm. For main culture for alcohol-fermentation, 1 L Erlenmeyer flask containing 495 mL of the medium [(yeast extract: 0.5 g, peptone: 0.5 g, MgSO₄: 0.1 g, K₂HPO₄: 0.1 g/100 mL (pH 5)] supplemented with 5 g of saccharified glucose obtained from wheat stalk powder and accessory nutrients was prepared and sterilized. 5 mL of the pre-culture solution was inoculated in the flask. Then, the flask was completely air-tight sealed, followed by shaking-culture at 30° C. with 140 rpm for 48 hours. To quantify alcohol in the culture fluid which was generated by alcohol-fermentation, the S. cerevisiae cells were eliminated by precipitation using centrifugation and supernatant was obtained. Absolute alcohol concentration (mL/mL) in the culture solution was analyzed by HPLC.

As a result, glucose yield was approximately 13% increased in the experimental group, compared with the negative control group not treated with Prx PP1084 protein (FIG. 3). When the ratio of chaperone protein to the enzyme cocktail was more than 1:1, glucose yield was approximately 6% increased, compared with the negative control. When the ratio was 1:2, glucose yield was approximately 13% increased (FIG. 4).

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended Claims.

What is claimed is:

1. A method for increasing saccharification efficiency of biomass including the step of treating chaperone protein and saccharogenic enzyme or saccharogenic enzyme composition together to biomass.

2. The method for increasing saccharification efficiency of biomass according to claim 1, wherein the biomass includes cellulose.

3. The method for increasing saccharification efficiency of biomass according to claim 1, wherein the chaperone protein is peroxiredoxin protein having the amino acid sequence represented by SEQ. ID. NO: 3.

4. The composition for increasing saccharification efficiency of biomass according to claim 1, wherein the saccharogenic enzyme is selected from the group consisting of cellulase, beta-glycosidase, and xylanase.

5. The method for increasing saccharification efficiency of biomass according to claim 1, wherein the saccharogenic enzyme or saccharogenic enzyme composition and the chaperone protein are treated at the ratio of 1:1˜1:5.

6. The method for increasing saccharification efficiency of biomass according to claim 1, wherein the saccharification process is performed at 30˜40° C. with pH 4.5˜6.

7. A method for mass-production of bioethanol from biomass comprising the following steps:
1) inducing saccharification of biomass by treating chaperone protein and saccharogenic enzyme or saccharogenic enzyme composition together to biomass; and
2) producing ethanol by alcohol-fermenting monosaccharides generated by the saccharification of step 1).

8. The method for mass-production of bioethanol from biomass according to claim 7, wherein the biomass of step 1) includes cellulose.

9. The method for mass-production of bioethanol from biomass according to claim 7, wherein the chaperone protein of step 1) is peroxiredoxin protein having the amino acid sequence represented by SEQ. ID. NO: 3.

10. The method for mass-production of bioethanol from biomass according to claim 7, wherein the saccharogenic enzyme or saccharogenic enzyme composition and the chaperone protein are treated at the ratio of 1:1˜1:5.

11. The method for mass-production of bioethanol from biomass according to claim 7, wherein the saccharogenic enzyme of step 1) is selected from the group consisting of cellulase, beta-glucosidase, and xylanase.

12. The method for mass-production of bioethanol from biomass according to claim 7, wherein the alcohol-fermentation of step 2) is performed in anaerobic condition at 30˜40° C. after treating monosaccharides with yeast.

13. A method for increasing saccharification efficiency of cellulose including the step of treating peroxiredoxin (Prx) protein, and a mixture of cellulase, beta-glycosidase, and xylanase to cellulose.

14. The method for increasing saccharification efficiency of cellulose according to claim 13, wherein the peroxiredoxin (Prx) protein was the amino acid sequence represented by SEQ. ID. NO: 3.

15. The method for increasing saccharification efficiency of cellulose according to claim 13, wherein the mixture comprises cellulase, beta-glycosidase, and xylanase mixed at the ratio of 6:1:1.

16. The method for increasing saccharification efficiency of cellulose according to claim 13, wherein peroxiredoxin (Prx) protein and the mixture of cellulase, beta-glycosidase, and xylanase are treated at the ratio of one of 1:0, 1:0.5, 1:1, 1:2, 1:3, 1:4, and 1:5.


SEQUENCE LISTING (3 sequences)

SEQ ID NO: 1 - DNA, 21 bp, Pseudomonas putida
ccgctcgagatgagcgtactc

SEQ ID NO: 2 - DNA, 22 bp, Pseudomonas putida
cgagctcttacagcttgccagc

SEQ ID NO: 3 - protein, 200 aa, Pseudomonas putida
MSVLVGKKAPDFTVPAVLGNGEIVDSFNLASAIKGKYGLVFFYPLDFTFVCPSELIALDNRIPDFQARNVEVIGVSIDSHFTHNAWRNTPVNNGGIGQVKYTLAADMTHEICKAYDVESEGGVAFRGAFLIDTNGVVRSQIVNDLPLGRNMDELLRLVDALQFHEEHGEVCPANWKKGDKGMNASPEGVAAYLSENAGKL